(19) United States
(12) Patent Application Publication
(10) Pub. No.: US 2004/0151729 A1
(43) Pub. Date: Aug. 5, 2004

(54) Novel long-term three-dimensional culture system

(76) Inventors: George Michalopoulos, Bethel, PA (US); William C. Bowen JR., White Oak, PA (US)

Correspondence Address: Deborah A. Somerville, KENYON & KENYON, One Broadway, New York, NY, 10004, US

(21) Appl. No.: 10/701,390

(22) Filed: Nov. 4, 2003

Related U.S. Application Data
(63) Continuation-in-part of application No. 10/281,575, filed on Oct. 28, 2002.

(51) Int. Cl. A61K 39/00; A61K 39/38

(52) U.S. Cl. 424/184.1

(57) ABSTRACT

The present invention relates to long-term, three-dimensional cultures of cells in a horizontally-rotating vessel and methods for producing the cultures and using the cultures as explants to treat various conditions.

### CROSS REFERENCE TO RELATED APPLICATIONS

This application claims the benefit of U.S. application Ser. No. 10/281,575 filed Oct. 28, 2002.

### BACKGROUND OF THE INVENTION

Conventional Tissue Cultures

Conventional culturing techniques generally do not produce tissue that could substitute for the same tissue in vivo. For instance, conventional culturing techniques involve either primary cultures or continuous cultures. Primary cultures are derived directly from excised, normal animal tissue and cultured either as an explant culture or dissociated cells in suspension. Primary cultures are not well suited for long-term culturing because they are labor intensive and can be maintained in vitro for only a limited time.

Continuous cultures are made of a single cell type that can be serially propagated in culture either indefinitely or for a limited number of cell divisions. In indefinitely propagating continuous cultures, the have generally undergone transformation into tumor cells or were derived from clinical tumors. Even though transformed cell lines may have limitless availability, they retain little of their original in vivo characteristics. Accordingly, continuous cultures are also not well suited as replacement tissues.

Generally, for both primary and continuous cultures, the tissue does not assume its in vivo histochemistry or morphology. This is partially due to the manner of culturing, which generally takes the form of either: (a) suspension (as single cells or small free-floating clumps in a solution media) or (b) a monolayer that is attached to the tissue culture flask. Additionally, the tissue may not assume the proper in vivo morphology because the culture may not have the proper extracellular support, cellular contacts and/or heterogeneous cellular profile. Thus, conventional culturing techniques are not well suited in providing tissue that could replace the same tissue in vivo or replace the function of that tissue in vivo.

Three-Dimensional Tissue Culturing

Three-dimensional tissue culturing techniques, such as the roller bottle method, provides tissue that can replace the same tissue in vivo or replace the function of that tissue in vivo. Roller bottles are cylindrical vessels that revolve slowly (between 0.25 and 25 revolutions per minute) and bathe the cells that are attached to the bottle with a suitable medium. Roller bottles are available typically with surface areas between 500-1000 cm².

One problem with the roller bottle method is the uneven attachment of some cell types to the bottle. This problem may be solved by optimizing the extracellular matrix proteins and cellular composition in the culture. Additionally, cells may attach evenly by optimizing the speed of rotation, generally by decreasing the speed, during the period of attachment.

Once the cells attach to the roller bottle, the culture develops the three-dimensional morphology and exhibits the tissue-specific gene expression observed in the tissue in vivo. Depending on the culture's cell type(s), different hormones and/or mitogens, or combinations thereof, may facilitate developing long-term, three-dimensional tissue cultures that could substitute for or replace the function of that tissue in vivo.

Three-Dimensional Hepatocyte/Nonparenchymal Cultures

While hepatocyte transplantation has been considered as an alternative to whole-organ transplantation, major technical barriers such as the inability to transfer donor hepatocytes into the liver of a recipient, in numbers to provide a beneficial result, have limited the usefulness of this approach. One of the major difficulties in constructing artificial liver tissue is that, to function effectively, the artificial liver tissue requires functionally active, differentiated hepatocytes present at high densities. Future success with artificial liver tissue will depend on the development of systems in which hepatocytes attached to matrices and packed at high density can retain long term their full functional capacity.

To generate artificial liver tissue, it will be necessary to provide in vitro cultures of hepatocytes. Unfortunately, one of the problems associated with the culturing of hepatocytes is that gene expression deteriorates rapidly as the hepatocytes proliferate. Likewise, long-term cultures of hepatocytes having stable gene expression can only be maintained in the absence of cell proliferation. Thus, one of the long-standing goals of culturing hepatocytes is the establishment of proliferating cultures with long-term gene expression.

A number of culture techniques have been developed that permit primary hepatocyte cultures to grow and/or express complex patterns of hepatocyte differentiation (Mitaka, et al., 1995, Biochem Biophys Res Commun 214: 310-317; Cable, 1997, Hepatology 26: 1444-1445; Block, et al., 1996, J. Cell Biol. 132: 1133-1149). Conditions have also been established that allow mature hepatocytes to enter into clonal expansion in cell culture (Block, et al., 1996, J. Cell Biol. 132: 1133-1149). For example, hepatocytes cultured in chemically defined hepatocyte growth medium (HGM) enter into DNA synthesis in response to polypeptide mitogens, notably epidermal growth factor (EGF), transforming growth factor-α (TGF-α), and hepatocyte growth factor (HGF). These mitogens induce multiple rounds of DNA synthesis and expansion of the cell population. The proliferating cells, however, lose most markers of hepatocyte differentiation while they retain expression of hepatocyte associated transcription factors HNF1, HNF4, and HNF3. In addition, proliferation of adult hepatocytes has been observed in serum-free medium supplemented with nicotinamide and epidermal growth factor (EGF) (Mitaka, T., et al., 1991, Hepatology 12: 21-30; Mitaka, T., et al., 1992, Hepatology 10:440-447; Mitaka, T., et al., 1993, J. Cell Physiol, 147: 461-468; Mitaka, T., et al., Cancer Res, 1993, 53: 3145-3148; Block, G. D., et al., 1996, J. Cell Biol. 132:1133-1149; Tateno, C., et al., 1996, Am J. Pathol 148: 383-392).

A number of devices which perform the function of the liver and involve blood perfusion have been described (Hagger et al., 1983, ASAIO J. 6:26-35; U.S. Pat. No. 5,043,260; U.S. Pat. No. 5,270,192: Demetriou et al., 1986, Ann. Surg 9:259-271). However, a number of problems are associated with the use of such devices for treatment of patients suffering from hepatic failure or dysfunction. Perhaps, the most significant problem is the inability to culture hepatocytes that retain hepatic function for prolonged periods of time, although, attempts have been made to circumvent this problem through the use of transformed hepatocytes that are capable of proliferating indefinitely (U.S. Pat. No. 4,853,324).

Just as it would beneficial to develop a support system to maintain hepatic functions and be useful in stabilizing patients in partial or complete hepatic failure, there is benefit to developing support systems to maintain adrenal, bile duct epithelial, corneal, pituitary and thyroid functions.

### SUMMARY OF THE INVENTION

In one aspect, the present invention features methods for cultures of small tissues, including of adrenal, bile duct epithelial, corneal, pituitary or thyroid tissues. In a preferred embodiment, the tissues are cultured in a horizontally-rotating vessel, such as a roller bottle, that is coated with extracellular matrix protein that facilitates attachment of the cells to the bottle. Further, the culture medium are enriched with growth agents, such as hormones, growth factors and/or cytokines, to promote differentiation and the histological and morphological development of the tissue culture, such that the culture resembles the tissue in vivo. An advantage to these cultures is that the cells display the function as endogenous cells.

In another aspect, the present invention features cultures of small tissues, including adrenal, bile duct epithelial, corneal, pituitary and thyroid tissues. In a preferred embodiment, the tissue is produced in a horizontally-rotating vessel, such as a roller bottle. The cells attach to extracellular matrix protein that coats the inner wall of the vessel. Further the tissue culture differentiates and assumes the histochemical and morphological profile as that tissue in vivo upon enrichment of the culture media with growth agents, such as hormones, growth factors and/or cytokines.

In yet another aspect, the present invention features therapeutic methods for using small tissue cultures to treat dysfunction of small tissues, including adrenal, bile duct epithelial, corneal, pituitary or thyroid tissues.

Other features and advantages of the invention will be apparent based on the following Detailed Description and claims.

### BRIEF DESCRIPTION OF THE DRAWINGS

FIG. 1 are light micrographs of hematoxylin and eosin-stained tissue sections from 20 day hepatocyte/nonparenchymal organoid cultures in roller bottles (A: original magnification, ×20; and B: original magnification, ×200).

FIG. 2 are electron micrographs of hepatocytes embedded in the tissue of the cultures showing: (a) on the left vacuolar inclusions (V) surrounded by collagenous matrix (Col.), round nuclei (N) and (b) on the right, at higher magnification, areas of cell-cell contact between differentiated hepatocytes, bile canaliculus (BC), desmosomes (D), tight junctions (TJ), glycogen (Gly), mitochondria (Mt) and rough endoplasmic reticulum (RER).

FIG. 3 are transmission electron micrographs of 30 day hepatocyte/nonparenchymal tissue, showing in A biliary epithelium (BE), tight junctions and desmosomes at cell-cell contacts (arrows) and basement membrane (BM); in B stellate cells (SC) with lipid droplet inclusions (arrows) and in C the endothelial cell (EC) and two lipid droplets (L) (scale bars: 1 μm (A and C), 2 μm (B)).

FIG. 4 are light micrographs of 20-day hepatocyte/nonparenchymal cultures maintained in complete medium with dexamethasone, HGF, and EGF, depicting the. FIG. 4A depicts the immunohistochemical staining cytokeratin 19 (FIG. 4A), desmin (FIG. 4B), the hepatocyte-specific HEPPAR antibody (FIG. 4C), coagulation factor VIII (FIG. 4D), cytochrome P-450 IIBI (FIG. 4E) and Mg++ ATPase (FIG. 4F) (H=hepatocytes and B=biliary epithelium).

FIG. 5 are light micrographs of histochemical staining of 20 day hepatocyte/nonparenchymal cultures depicting PCNA stain of an organoid ribbon (FIG. 5A) and immunohistochemical stain for Ki-67 (FIG. 5B; original magnifications, ×200).

FIG. 6 are light micrographs of hematoxylin and eosin-stained 25 day hepatocyte/nonparenchymal cultures, wherein the cultures were incubated with either: dexamethasone (Dex), hepatocyte growth factor (HGF) and epidermal growth factor (EGF) (FIG. 6A), HGF and EGF (FIG. 6B), Dex (FIG. 6C), or neither Dex, HGF nor EGF (FIG. 6D; arrows point to two mitoses; original magnifications, ×200).

FIG. 7 are light micrographs of the immunohistochemistry for cytokeratin 19 in 25 day hepatocyte/nonparenchymal cultures, wherein the cultures were incubated with either Dex, HGF and EGF (FIG. 7A), HGF and EGF (FIG. 7B), Dex (FIG. 7C), or neither Dex, HGF nor EGF (FIG. 7D; original magnifications, ×200).

FIG. 8 are autoradiograms of Northern blots for either albumin, TGF-β, collagen type IV or GAPDH in hepatocyte/nonparenchymal cultures at different days (8 or 23 days), maintained in the presence of either HGF or EGF or both (NRL=whole normal rat liver tissue).

### DETAILED DESCRIPTION OF THE INVENTION

Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, an element means one element or more than one element.

“Adrenal tissue” is used herein to refer to both the adrenal cortex and adrenal medulla. The cells of the adrenal medulla secrete amine hormones, such as epinephrine (E) and norepinephrine (NE), and the cells of the adrenal cortex secrete steroid hormones, such as aldosterone (also known as mineralocorticoid), androstenedione, dehydroepiandrosterone, cortisol and corticosterone. Depending upon exposure to neuronal stimulation, hormones, ions or organic nutrients, these conditions affect the function of adrenal tissue either by stimulating or inhibiting the secretion of hormones from the adrenal tissue. For example, adrenocorticotropic hormone (ACTH) induces secretion of cortisol from the adrenal cortex.

Adrenal tissue cultures may be used to correct problems in organic metabolism, stress responses, immune function, sex drive in women and the kidney's excretion of sodium, potassium and acid. Such conditions may be associated with adrenal insufficiency and related to Addison's Disease. One of skill in the art would recognize conditions for which adrenal tissue would be useful.

Adrenal cells may be cultured in roller bottles in the medium as described in Levi A, et al. Science 229: 393-395, 1985; Greene L A, Tischler A S., Proc. Natl. Acad. Sci. USA 73: 2424-2428, 1976; Biocca S, et al, EMBO J. 2: 643-648, 1983, Weber E, et al., J. Biol. Chem. 271: 6963-6971, 1996; Yasumura Y, et al., Cancer Res. 26: 529-535, 1966. To stimulate development of adrenal cortex, transforming growth factor-β (TGFβ) and adrenocorticotropin hormone (ACTH) may be added to the culture medium. To stimulate the development of adrenal medulla, nerve growth factor (NGF) may be added to the culture medium.

“Bile duct” is used herein to refer to the collection of bile canaliculi that converge to form the common hepatic duct that transfers bile from the liver to the gallbladder. Specifically, bile duct epithelia tissue is used herein to refer to the cells that develop into bile ducts and perform the following: (a) transfer of bile from the liver to the gall bladder and (b) secrete of a bicarbonate-rich salt from the epithelial cells that helps to neutralize acid in the duodenum. Upon differentiation bile duct epithelial cultures should form multicellular ducts, high expression of γ-glutamyl transpeptidase, and the production of cilia which grow into the ductal lumen. Bile duct epithelial tissue may be used to correct problems with the bile duct, such as sclerosing cholangitis, biliary cirrhosis and biliary atresia. One of skill in the art would recognize conditions for which bile duct epithelial tissue would be useful. Bile duct epithelial cells may be cultured in roller bottles in the medium as described in Matsumoto, K. et al., Hepatology 20: 376-382, 1994. To stimulate the formation of bile ducts, hepatocyte growth factor and epidermal growth factor may be added to the culture medium.

“Corneal tissue” is used herein to refer to the collection of cell that: (a) coat the outer portion of the eye to provide a physical barrier that shields the inside of the eye from germs, dust, and other harmful matter, (b) transfer water and ions from the stroma into the conjunctival sac of the eye, (c) synthesize of proteins that may maintain the Descemet's membrane of the eye and (d) provide high refraction of light into the eye so that an image is focused on the retina.

Corneal tissue may be used to correct problems with structure or function of the eye, such as myopia, hyperopia, astigmatism, corneal dystrophy and Steven-Johnson Syndrome. One of skill in the art would recognize conditions for which corneal tissue would be useful.

The corneal tissue should exhibit the proper permeability of intact corneal epithelia as described in Tchao (Alternative Methods of Toxicology, 1988, Vol. 6, pp. 271-283, Goldberg, A. M., ed. Mary Ann Liebert, Inc., New York, N.Y.), L. Ubels, et al. (Toxicology in Vitro 16 (5) (2002) pp. 621-628.), L. H. Bruner, et al (Toxicology in Vitro 12 (6) (1998) pp. 669-690).

Corneal cells may be cultured in roller bottles in the medium described in U.S. Pat. Nos. 5,585,265 and 5,672,498. To stimulate the formation of corneal tissue epidermal growth factor may be added to the culture medium.

“Extracellular matrix protein” is used herein to refer to glycoprotein, proteoglycans, complex carbohydrates and other molecules that serve the following functions: (a) providing structural support, tensile strength or cushioning, (b) providing substrates and pathways for cell adhesion and cell migration and (c) regulating differentiation and metabolic function in a direct or indirect fashion, i.e., by modulating cell growth by binding growth factors. Extracellular matrix protein is intended to encompass, i.e., collagen type I, II, III, IV, V, VI, VII, VIII, IX, X, XI, XII, XIII, XIV, XV, XVI and XVII; elastin; fibronectin; laminin; proteoglycans that include one or more glycosaminoglycan side chains, such as heparan sulphate, heparin, chondroitin sulphate, dermatan sulphate, keratan sulphate and hyaluronic acid; vitronectin; thrombospondin; tenascin (cytotactin); entactin (nidogen); osteonectin (SPARC); anchorin CII; chondronectin; link protein; osteocalcin; bone sialoprotein; osteopontin; epinectin; hyaluronectin; amyloid P component; fibrillin; merosin; s-laminin; undulin; epiligrin and kalinin. Other extracellular matrix proteins are described in Hay, E. D. (Ed.) (1991) Cell Biology of Extracellular Matrix Proteins, 2^{nd} ed (Plenum, N.Y.) and Sandell, L J & Boyd, CD (Eds) (1990) Extracellular Matrix Genes (Academic Press, New York).

“Growth agent” is used herein to refer to compounds that control the growth, differentiation and maintenance of tissue form and function, and is intended to encompass serum, hormones, growth factors and cytokines, such as aldosterone, androstenedione, dehydroepiandrosterone, cortisol, corticosterone, growth hormone (GH), thyroid stimulating hormone (TSH), adrenocorticotrophin (ACTH), prolactin, follicle-stimulating hormone (FSH), luteinizing hormone (LH), β-lipotropin, β-endorphin, acidic fibroblast growth factor (FGF-1); activin; angiogenin; astroglial growth factor-i and -2 (AGF-1 and AGF-2); basic fibroblast growth factor (FGF-2); brain-derived neurotrophic growth factor (BDNF); transforming growth factor α (TNF-α); cholera toxin (CT); ciliary neurotrophic factor (CNTF); endothelial cell growth factor (ECGF); enothelial growth supplement (ECGS); endotoxin; epidermal growth factor (EGF); erythropoietin (EPO); eye-derived growth factor-1 and -2 (EDGF-1 and EDGF-2); fibroblast growth factor-3, -4, -5, -6, -9 (FGF-3, FGF-4, FGF-5, FGF-6, FGF-9); granulocyte colony-stimulating factor (G-CSF); granulocyte/macrophage colony-stimulating factor (GM-CSF); heparin-binding epidermal growth factor (HB-EGF); hepatocyte growth factor (HGF); heregulin (HRG); macorphoge-activating factor (MAF); insulin (Ins); insulin-like growth factor-1 and -2 (IGF-1 and IGF-2); interferon-α1 and -α2 (INF-α1 and INF-α2); interferon-β and β2 (INF-β and INF-β2); interferon γ (INF-γ); interleukin-1, -2, -3, 4, -5, -6, -7, 8, -9, -10, -11 and -12 (IL-1, IL-2, IL-3, IL-4, IL-5, IL-6, IL-7, IL-8, IL-9, IL-10, IL-11 and IL-12); keratinocyte growth factor (KGF); Leukemia inhibitory factor (LIF); lipopolysaccharide (LPS); macrophage inflammatory protein-1α (MIP-1α); monocyte/macrophage colony-stimulating factor (M-CSF); mullerian inhibition factor (MIF); nerve growth factor (NGF); oncostatin M (OSM); phytohemagglutinin (PHA); platelet-derived endothelial cell growth factor (PD-ECGF); platelet-derived growth factor family, including PDGF-A and PDGF-B and vascular endothelial cell growth factor (VEGF); phorbol meristate acetate (PMA); pokeweed mitogen (PWM); stem cell factor (SCF); transferrin (Tfn) and transforming growth factor α and β (TGF-α, TGF-β1, TGF-β2, TGF-β3, TGF-β4, TGF-β5 and TGF-β6). Those of skill in the art will also recognize that one or more commercially available substances may be used as additives or substitutions to the medium to support the growth of stem cells.

“Horizontally-rotating vessel” is used herein to refer to a container that rotates along its horizontal axis, such as a roller bottle as described in U.S. Pat. No. 4,962,033 and Michalopoulos, G K, et al., (2001) Am. J. Path. 159:1877-1887). The vessel may contain pleats that facilitate cell adhesion and growth. The speed of rotation may vary to conform to the metabolic requirement of the cells in the vessel such that the speed of rotation is between 0.25-25 rotations per minute. Roller bottles are available typically with surface areas between 500-1000 cm².

“Pituitary tissue” is used herein to refer the cells of both the anterior and posterior pituitary. The cells of the anterior pituitary secrete growth hormone (GH, aslo know as somatropin), thyroid stimulating hormone (TSH, also known as thyrotropin), adrenocorticotrophin (ACTH), prolactin, follicle-stimulating hormone (FSH), luteinizing hormone (LH), β-lipotropin and β-endorphin. The cells of the posterior pituitary secrete oxytocin and vasopressin (also known as antidiuretic hormone, ADH). Depending upon exposure to neuronal stimulation, hormones, ions or organic nutrients, this exposure either stimulates or inhibits secretion of hormones from the cells of either the anterior or posterior pituitary. For example, corticotropin-releasing hormone (CRH) induces the release of ACTH from the anterior pituitary. Conversely, prolactin-inhibiting factor inhibits the release of prolactic from the anterior pituitary.

Pituitary tissue may be used to correct problems with growth, functioning of the thyroid and adrenal glands, the development of secondary sex characteristics, breast milk synthesis, the kidney's water secretion, blood pressure, uterine motility, gamete production and the gonad's sex hormone secretion. These conditions may result from hyperpituitarism or hypopituitarism, and may be associated with acromegaly, galactorrhea, amenorrhea, Cushing's Syndrome, Nelson's Syndrome, Sheehan's Syndrome and Syndrome of Inappropriate ADH (SIADH). One of skill in the art would recognize conditions for which pituitary tissue would be useful.

Cells of pituitary tissue may be cultured in roller bottles in medium described in Hurbain-Kosmath I, et al., In Vitro Cell. Dev. Biol. 26: 431-440, 1990; Yasamura Y., Science 154: 1186-1189, 1966; and Tashjian A H Jr, et al., Endocrinology 82: 342-352, 1968. To stimulate formation of pituitary tissue, growth hormone may be added to the tissue.

“Small tissues” is used herein to refer to tissues weighing less than 10 grams, such as the adrenal gland, bile duct epithelia, cornea, pituitary gland and the thyroid gland.

“Thryoid tissue” is used herein to refer to the cells of the thyroid that function by secreting thyroid hormones, such as thyroxine (T₄) and thriiodothyronine (T₃), and calcitonin. Cells of thyroid tissue secrete these hormones upon exposure to neuronal stimulation, hormones, ions or organic nutrients. For example, thyroid stimulating hormone (TSH) induces secretion of T₃ and T₄ from thyroid cells. Alternatively, cells of thyroid tissue take up iodide, is used in the production of thyroid hormones. Thyroid tissue cultures may be used to correct problems in metabolic function, growth, brain development and function and plasma calcium levels. Thyroid pathologies, such as goiter, Grave's disease, Hashimoto's disease, adenomas and carcinomas, involve impairment of thyroid function and, typically, excision of the thyroid itself. These conditions may be treated with the thyroid tissues reported herein. Also, one of skill in the art would recognize conditions for which thyroid tissue would be useful. Thyroid cells may be cultured in roller bottles in medium described in Curcio, F. et al., Proc. Natl. Acad. Sci. USA 91: 9004-9008, 1994. To stimulate the formation of thyroid tissue, extracts from the hypothalamus and pituitary may be added to the medium. These extracts are described in Coon et al., Proc. Natl. Acad. Sci. USA 86: 1703-1707, 1989 and Wolozin, B. et al., J. Mol. Neurosci. 3: 137-146, 1992.

“Tissue culture” is used herein to refer to a method of growing both undisaggregated fragments of tissue and disaggregated tissue ex vivo, and is intended to encompass organ, cell, histotypic and organotypic cultures. The term “organ culture” is used to refer to a three-dimensional culture of undisaggregated tissue retaining some or all of the histological features of the tissue in vivo. “Cell culture” is used herein to refer to a culture derived from dispersed cell taken from original tissue, from a primary culture, or from a cell line or cell strain by enzymatic, mechanical, or chemical disaggregation. “Histotypic culture” is used herein to refer to cells that have been reaggregated to re-create a three-dimensional tissue-like structure, i.e., by cultivation at high density in a filter well, perfusion and overgrowth on a monolayer in a flask or dish, reaggregation in suspension over agar or in real or simulated zero gravity or infiltration of a three-dimensional matrix such as collagen gel. “Organotypic culture” is used herein to refer to recombining cells of different lineages and reaggregated those different cell types to re-create a three-dimensional tissue-like structure, i.e., by cultivation at high density in a filter well, perfusion and overgrowth on a monolayer in a flask or dish, reaggregation in suspension over agar or in real or simulated zero gravity or infiltration of a three-dimensional matrix such as collagen gel.

Culturing Methods

Adrenal, bile duct epithelial, corneal, pituitary and thyroid tissue can be dissected and treated with enzymes to disperse the tissue into a suspension of cells. Such enzymes include, but are not limited to, trypsin, chymotrypsin, collagenase, elastase and/or hylauronidase. After dispersion, the cells can be incubated in a horizontally-rotating vessel that is coated with extracellular matrix protein. Further, the medium may be enriched in various growth agents that promote the differentiation of the various cell types. Different media could include but are not limited to balanced salts solution such as Hank's Balanced Salt Solution (HBSS), any complete tissue culture media such as Minimal Essential Medium (MEM), Dulbecco's Minimal Essential Medium (DMEM), Ham's Medium F12, etc. Examples 1-6 describe culturing of adrenal, bile duct epithelia, corneal epithelial, pituitary and thyroid tissue.

Detection of Various Cell Types in Culture

Differentiated cells in the tissue cultures may be detected using tissue-specific markers by immunological techniques, such as immunohistochemistry (for example, of fixed cells or tissue sections) for intracellular or cell-surface markers, Western blot analysis of cellular extracts, and enzyme-linked immunoassay, for cellular extracts or products secreted into the medium. The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard amplification methods.

Alternatively, differentiated cells may be detected using selection markers. For example, either before or during the incubation of the cells in a “horizontally-rotating vessel” the cells can be stably transfected with a marker that is under the control of a tissue-specific regulatory region as an example, such that during differentiation, the marker is selectively expressed in the specific cells, thereby allowing selection of the specific cells relative to the cells that do not express the marker. The marker can be, e.g., a cell surface protein or other detectable marker, or a marker that can make cells resistant to conditions in which they die in the absence of the marker, such as an antibiotic resistance gene (see e.g., in U.S. Pat. No. 6,015,671).

Administering Tissue Cultures

Compositions comprising tissue cultures may be administered to a subject to provide various cellular or tissue functions.

Such compositions may be formulated in any conventional manner using one or more physiologically acceptable carrier optionally comprising excipients and auxiliaries which facilitate processing of the compounds into preparations which can be used pharmaceutically. Proper formulation is dependent upon the route of administration chosen. The compositions may be packaged with written instructions for use of the cells in tissue regeneration, or restoring a therapeutically important metabolic function. Tissue cultures may also be administered to the recipient in one or more physiologically acceptable carriers. Carriers for these cultures may include, but are not limited to, solutions of phosphate buffered saline (PBS) or lactated Ringer's solution containing a mixture of salts in physiologic concentrations.

Tissue cultures may be administered by injection into a target site of a subject, preferably via a delivery device, such as a tube, e.g., catheter. In a preferred embodiment, the tube additionally contains a needle, e.g., a syringe, through which the tissue can be introduced into the subject at a desired location. Specific, non-limiting examples of administering tissues to subjects may also include administration by subcutaneous injection, intramuscular injection, or intravenous injection. If administration is intravenous, an injectible liquid suspension of tissue can be prepared and administered by a continuous drip or as a bolus. As used herein, the term “solution” includes a pharmaceutically acceptable carrier or diluent in which the tissue of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating tissue cultures as described herein, in a pharmaceutically acceptable carrier or diluent and, as required, other ingredients enumerated above, followed by filter sterilization.

The tissue culture may be administered in any fashion as previously discussed, for example in a dose of, for example 0.25-1.0×10⁶ cells. Different dosages can be used depending on the clinical circumstances. The tissue cultures may be administered systemically (for example intravenously) or locally (for example by direct application under visualization during surgery). For such injections, the tissue cultures may be in an injectible liquid suspension preparation or in a biocompatible medium which is injectible in liquid form and becomes semi-solid at the site of damaged tissue. A conventional intra-cardiac syringe or a controllable endoscopic delivery device can be used so long as the needle lumen or bore is of sufficient diameter (e.g. 30 gauge or larger) that shear forces will not damage the culture that being delivered.

Tissue cultures may be administered in a manner that permits them to graft to the intended tissue site and reconstitute or regenerate the functionally deficient area. The tissue cultures may be administered directly, or as part of a bioassisted device that provides temporary or permanent organ function.

Genetic Engineering of the Cells in the Tissue Cultures

Cells of the tissue culture may be genetically engineered to produce a particular therapeutic protein. As used herein the term “therapeutic protein” includes a wide range of biologically active proteins including, but not limited to, growth factors, enzymes, hormones, cytokines, inhibitors of cytokines, blood clotting factors, peptide growth and differentiation factors. Particular differentiated cells may be engineered with a protein that is normally expressed by the particular cell type. For example, adrenal cells can be engineered to produce steroid hormones.

Methods which are well known to those skilled in the art can be used to construct expression vectors containing a nucleic acid encoding the protein of interest linked to appropriate transcriptional/translational control signals. See, for example, the techniques described in Sambrook, et al. Molecular Cloning, A Laboratory Manual, Cold Spring Harbor Laboratory, N.Y. (1992) and Ausebel et al. Current Protocols in Molecular Biology, Greene Publishing Associates & Wiley Interscience, N.Y (1989).

Suitable methods for transferring vector or plasmids into the cells of the tissue cultures include lipid/DNA complexes, such as those described in U.S. Pat. Nos. 5,578,475; 5,627,175; 5,705,308; 5,744,335; 5,976,567; 6,020,202; and 6,051,429. Suitable reagents include lipofectamine, a 3:1 (w/w) liposome formulation of the poly-cationic lipid 2,3-dioleyloxy-N-[2(sperminecarbox-amido)ethyl]-N,N-dimethyl-1-propanaminium trifluoroacetate (DOSPA) (Chemical Abstracts Registry name: N-[2-(2,5-bis[(3-aminopropyl)amino]-1-oxpentyl}amino) ethyl]-N,N-dimethyl-2,3-bis(9-octadecenyloxy)-1-propanamin-trifluoroacetate), and the neutral lipid dioleoyl phosphatidylethanolamine (DOPE) in membrane filtered water. Exemplary is the formulation Lipofectamine 2000TM (available from Gibco/Life Technologies # 11668019). Other reagents include: FuGENE™ 6 Transfection Reagent (a blend of lipids in non-liposomal form and other compounds in 80% ethanol, obtainable from Roche Diagnostics Corp. # 1814443); and LipoTAXI™ transfection reagent (a lipid formulation from Invitrogen Corp., produce the desired biologically active protein. #204110). Transfection of the cells in the tissue culture can be performed by electroporation, e.g., as described in Roach and McNeish (Methods in Mol. Biol. 185:1 (2002)). Suitable viral vector systems for producing cells with stable genetic alterations may be based on adenoviruses, lentiviruses, retroviruses and other viruses, and may be prepared using commercially available virus components.

Exemplifications

The invention, having been generally described, may be more readily understood by reference to the following examples, which are included merely for purposes of illustration of certain aspects and embodiments of the present invention, and are not intended to limit the invention in any way.

### EXAMPLE 1

### Histological Organization in Hepatocyte Organoid Cultures

The purpose of the present example is the demonstration that cultures of hepatocytes grown in chemically defined hepatocyte growth medium (HGM) containing hepatocyte growth factor and epidermal growth factor and dexamethasone retain their hepatic functions while maintaining their capacity to proliferate.

Materials and Methods

Materials

Male Fischer 344 rats from Charles River (Wilmington, Mass.) were used for the studies described below. All animals were treated according to protocols approved by the animal care institutional review board.

EGF was obtained from Collaborative Biomedical (Waltham, Mass.). Collagenase for hepatocyte isolation was obtained from Boehringer Mannheim (Mannheim, Germany). Vitrogen (Celtrix Labs., Palo Alto, Calif.) was used for collagen coating of roller bottles. General reagents were obtained from Sigma Chemical Co. (St. Louis, Mo.). EGF was purchased from BD Pharmingen (San Diego, Calif.). HGF used for these studies was the)5 variant and was kindly donated by Snow Brand Co. (Toshigi, Japan). Antibodies were obtained from the following sources: proliferating cell nuclear antigen (PCNA) from Signet Laboratories (Dedham, Mass.); Ki-67 from Santa Cruz Biologicals (Santa Cruz, Calif.); desmin, cytokeratin 19, HEPPAR, and factor VIII from DAKO Corp (Carpinteria, Calif.).

Immunocytochemistry

Tissues from the cultures were harvested and fixed in 10% formalin. Tissues were paraffin-embedded, sectioned at 4 to 5 μm, and affixed to charged slides (Superfrost/Plus; Fisher Scientific, Pittsburgh, Pa.). Immunohistochemistry was performed using the Vectastain ABC Elite kit (Vector Laboratories, Inc., Burlingame, Calif.). PCNA antibody was used at a concentration of 1:100 on sections that were microwaved in citrate buffer. Ki-67 antibody was used at a concentration of 1:200 and sections were heated under pressure in citrate buffer. Desmin antibody was used at a concentration of 1:100. Cytokeratin 19 antibody was used at 1:10 in sections microwaved in citrate buffer. HEPPAR antibody was used at a concentration of 1:25 in sections microwaved in citrate buffer. Factor VIII antibody was used at 1:400 sections that were treated with pepsin. Secondary antibodies used for this project were goat anti-rabbit, goat anti-mouse, and donkey anti-goat (Chemicon, Temecula, Calif.) all used at a 1:500 dilution.

Isolation and Culture of Hepatic Cell Populations

Rat hepatocytes were isolated by an adaptation of Seglen's calcium two-step collagenase perfusion technique (Seglan P O, 1976, Methods Cell Biol 13:29-83) as previously described from our laboratory (Michalopoulos G K, 1999 Hepatology 29:90-100). Hepatocytes isolated from collagenase perfusion of rat liver were added at a concentration of 210,000,000 hepatocytes per 250 ml of medium. As previously described, these preparations are known to contain contaminant small numbers of other hepatic cellular elements, including stellate cells, Kupffer cells, and very few bile duct epithelial cells. The latter typically do not comprise >0.05% of the inoculated cell population (Seglan P O, 1976, Methods Cell Biol 13:29-83). By hematoxylin and eosin (H&E) stain of smears of the isolated hepatocyte pellet, small cells arranged in a ductular configuration were occasionally noted. Although precise calculations were difficult to obtain given the random distribution of these clusters, their number seemed to be even less than the range for ductular cell contamination previously described.

The supernatant of the first low-gravity centrifugation used to prepare hepatocytes was subjected to a 1000×g centrifugation for 3 minutes. This fraction primarily contains stellate cells, bile duct cells, and endothelial cells. Small hepatocytes are also present in this fraction, typically comprising ˜5% of the cells.

Freshly isolated hepatocytes were added to roller bottles (850 cm² surface) obtained from Falcon (Franklin Lakes, N.J.). Each bottle contained 210,000,000 freshly isolated hepatocytes in 250 ml of HGM medium supplemented with HGF (20 ng/ml) and EGF (10 ng/m) (Block G D et al., 1996, J Cell Biol 132:1133-1149). The bottles were rotated at a rate of 2.5 rotations per minute and kept in an incubator maintained at 37° C., saturated humidity, and 5% CO2.

HGM medium was prepared as previously described (Block G D et al., 1996, J Cell Biol 132:1133-1149). Dulbecco's modified Eagle's medium powder, HEPES, glutamine, and antibiotics were purchased from Life Technologies, Inc., Grand Island, N.Y. ITS mixture (insulin, transferrin, selenium) was purchased from Boehringer Mannheim. All other additives were cell-culture grade (Sigma). Unless otherwise indicated for specific experiments, the basal HGM consisted of Dulbecco's modified Eagle's medium supplemented with purified bovine albumin (2.0 g/L), glucose (2.0 g/L), galactose (2.0 g/L), omithine (0.1 g/L), proline (0.030 g/L), nicotinamide (0.305 g/L), ZnCl2 (0.544 mg/L), ZnSO4:7H20 (0.750 mg/L), CuSO4:5H20 (0.20 mg/L), MnSO4 (0.025 mg/L), glutamine (5.0 mmol/L), and dexamethasone (10-7 mol/L). Penicillin and streptomycin were added to the basal HGM at 100 mg/L and 100 μg/L, respectively. The mixed basal HGM was sterilized by filtration through a 0.22 μm low-protein-binding filter system, stored at 4° C., and used within 4 weeks. ITS (1.0 g/L) (rh-insulin 5.0 mg/L, human transferrin 5.0 mg/L, 30% diferric iron saturated, and selenium 5.0 μg/L) was added after filtration immediately before use. The growth factors, as required, were added to HGM fresh at the specified concentrations every time the medium was changed.

Transmission Electron Microscopy

Samples for transmission electron microscopy were washed once in phosphate-buffered saline (PBS) with 1 mmol/L MgCl2, 0.5 mmol/L CaCl2, then fixed overnight at 4° C. in 2.5% glutaraldehyde in PBS. Samples were washed three times with PBS then postfixed in 1% OsO4, 1% KFe(CN)6 in PBS for 1 hour at room temperature. Samples were washed three times in PBS, then dehydrated through graded series (30 to 100%) of ethanol. After three changes of 100% ethanol, samples were infiltrated with several changes of Polybed 812 resin (Polysciences, Warrington, Pa.) at room temperature, with a change overnight at 4° C. Thick sections (300 μm), obtained using a Reichert (Vienna, Austria) ultramicrotome fitted with a diamond knife, were heated onto glass slides, stained with 1% Toluidine blue, and rinsed with water. Ultrathin sections (60 nm) were collected on Formvar-coated (Fullam, Schenectady, N.Y.) grids and stained with 2% uranyl acetate in 50% methanol for 10 minutes, then 1% lead citrate for 7 minutes. Sections were analyzed and photographed on a JEOL JEM 1210 transmission electron microscope at 80 kV.

Analysis of Gene Expression by Northern Blots

Total RNA was extracted by use of RNAzol B (BioTECX, Houston, Tex.). RNA extraction from roller-bottle cultures was performed by mixing 1 volume (pelleted) of scraped tissues with three volumes of RNAzol. RNA was purified according to the manufacturer's guidelines. RNA concentration and purity were determined by routine spectrophotometry. Size separation of 20 μg of RNA per lane was completed on denaturing 1% agarose gels and transferring to nylon membranes (Amersham, Piscataway, N.J.) by the capillary method. After cross-linking under ultraviolet light, membranes were hybridized overnight with specific complementary DNA that had been labeled with a [32P]dCTP using an Amersham random primer kit. Membranes were subsequently washed under high stringency conditions and exposed to R film (photographic film) (Eastman-Kodak, Rochester, N.Y.) for 1 to 3 days. Quantification of the RNA hybridization bands was performed by laser densitometry.

Collagen probes were obtained from ATCC (Rockville, Md.). Rat albumin probe was obtained from Dr. Mark Zern; transforming growth factor (TGF)-β1 human probe from Dr. Derynck; Cytochrome P-450 IIB 1 (mouse) from Dr. Negishi; collagen IV (mouse) from ATCC.

Results

Culture Conditions and Basic Histology

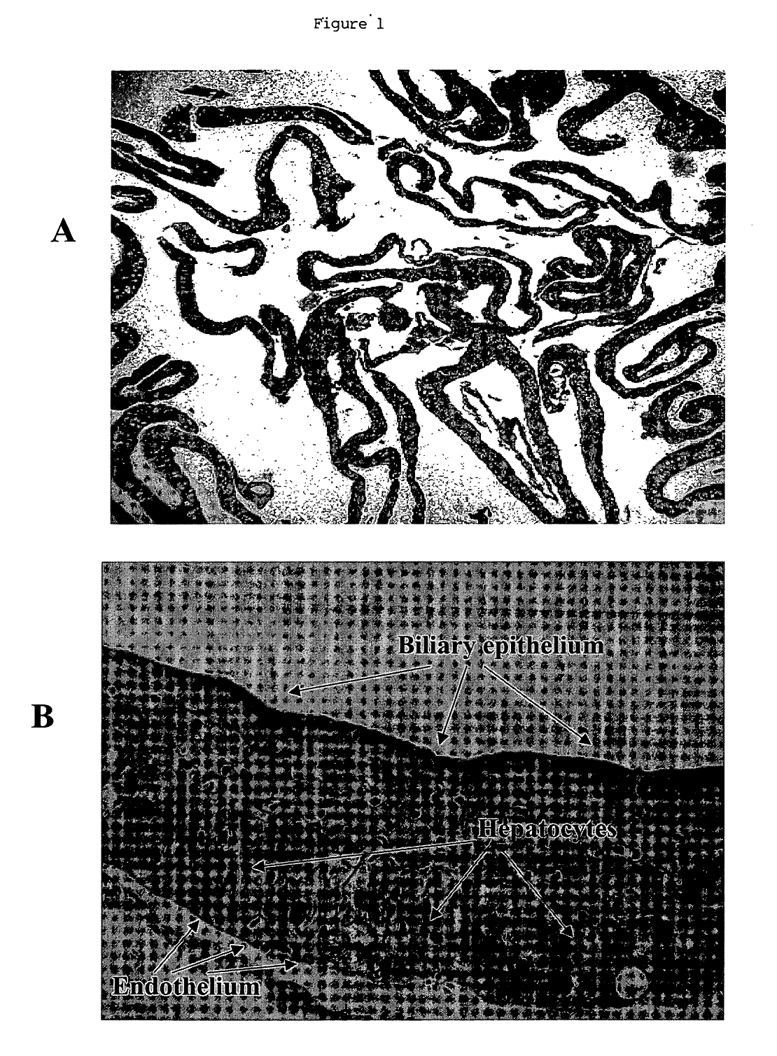

The surface of the pleated roller bottles was coated with collagen type I before inoculation of cells, as previously described (Strom S C and Michalopoulos G, 1982, Methods Enzymol 82:544-555). The culture medium HGM was supplemented with HGF and EGF unless otherwise indicated for specific experiments. The inoculated cells attach to the surface of the culture bottle within ˜24 hours. Approximately 50% of the hepatocytes enter into apoptosis in the first 5 days of the culture. The apoptotic cells gradually disappear from the mix later on as connective tissue develops. By day 18 to 20 of the cultures, the organization of the cellular elements acquires its typical configuration. Sheets of tissue of gray-brown coloration cover the surface of the roller bottle, being more prominent in the grooves of the internal surface. Approximately 2 to 4 g of tissue can be recovered from a roller bottle at 30 days in culture. The sheets of tissue were scraped from the surface of the roller bottles, pelleted, and processed as necessary for histological and biochemical evaluations. The observed histology is standard and highly reproducible. FIG. 1A is a low-power (×20) view of the histological appearance of the many ribbons of tissue removed by scraping from the roller bottle. A higher power view (×200) is shown in FIG. 1B. Each ribbon is composed of the same standard histology. On the surface facing the medium there is a continual monolayer of cuboidal biliary epithelium. Below the biliary layer there is a 5 to 10 cell layer composed of hepatocytes embedded in connective tissue elements. There is a variable amount of connective tissue separating hepatocytes from the biliary layer, from complete absence to a thick layer separating the two cell types (as shown in FIGS. 1A-B). Hepatocytes have a variable nuclear and nucleolar structure, suggesting different degrees of ploidy. Attached to the substrate and underlying the hepatocytes and connective tissue is a layer of endothelial cells. This typical morphology is seen when the hepatocyte cell fraction from the collagenase perfusion is placed in culture. When the nonparenchymal cell pellet (containing endothelial cells, stellate cells, and occasional small hepatocytes) is put in culture under similar conditions, no growth was observed (data not shown).

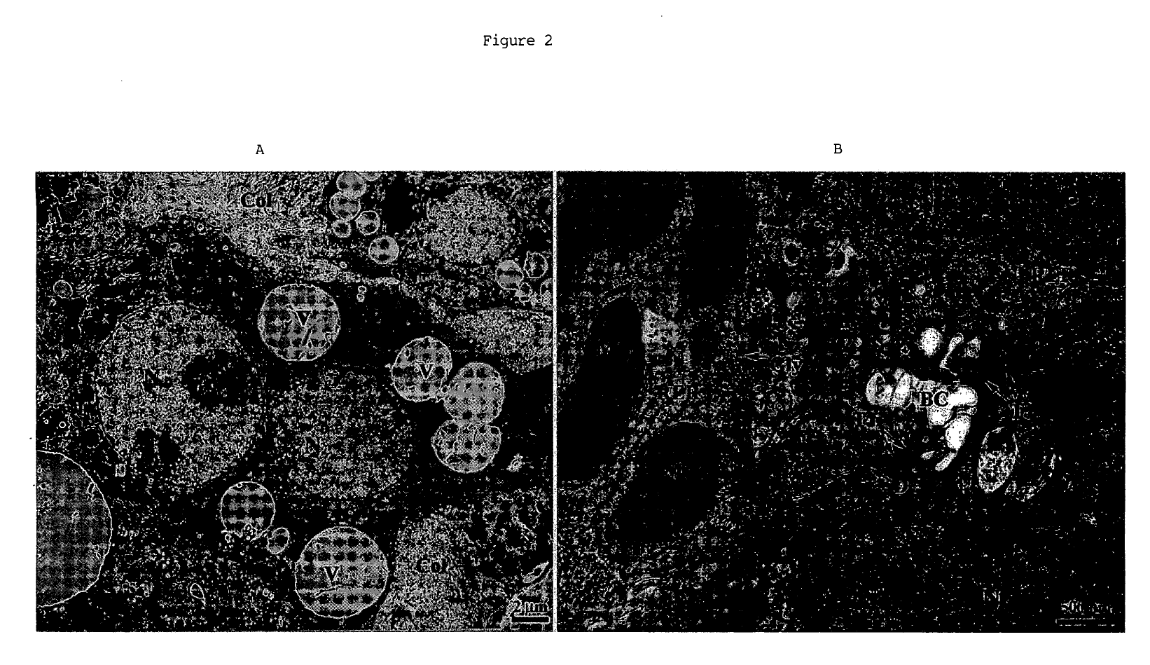

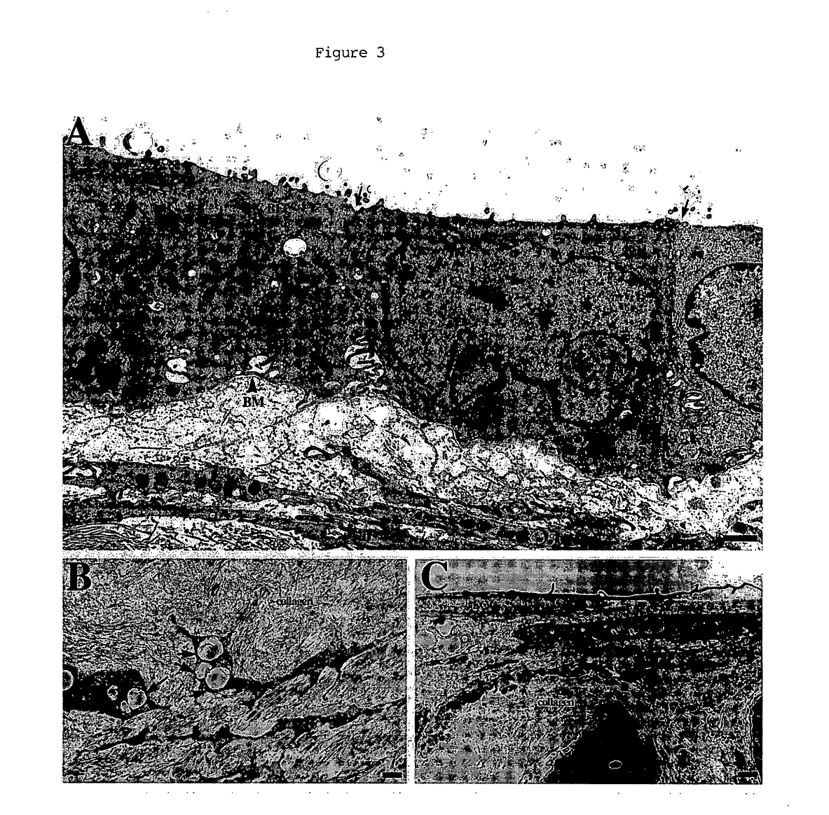

By electron microscopy, all typical features of the cellular elements present are easily identified. FIG. 2A shows a binucleate hepatocyte. Details of cytoplasmic organization including mitochondria, rough endoplasmic reticulum, bile canaliculi, tight junctions, and so forth, are shown in FIG. 2B. FIG. 3 shows the cellular ultrastructure of other cellular elements of the organoid cultures. The biliary epithelium (FIG. 3A) displays typical cerebriform nuclei and surface microvilli. A dense network of collagen fibrils underlies the surface epithelium. Stellate-like cells with small lipid droplets are shown embedded in the connective tissue matrix in FIG. 3B. Endothelial cells at the basal layer also display typical subcellular architecture for the cell type (FIG. 3C). The presence of fenestrated endothelium was not detected. Occasional macrophages were also seen.

Histochemistry

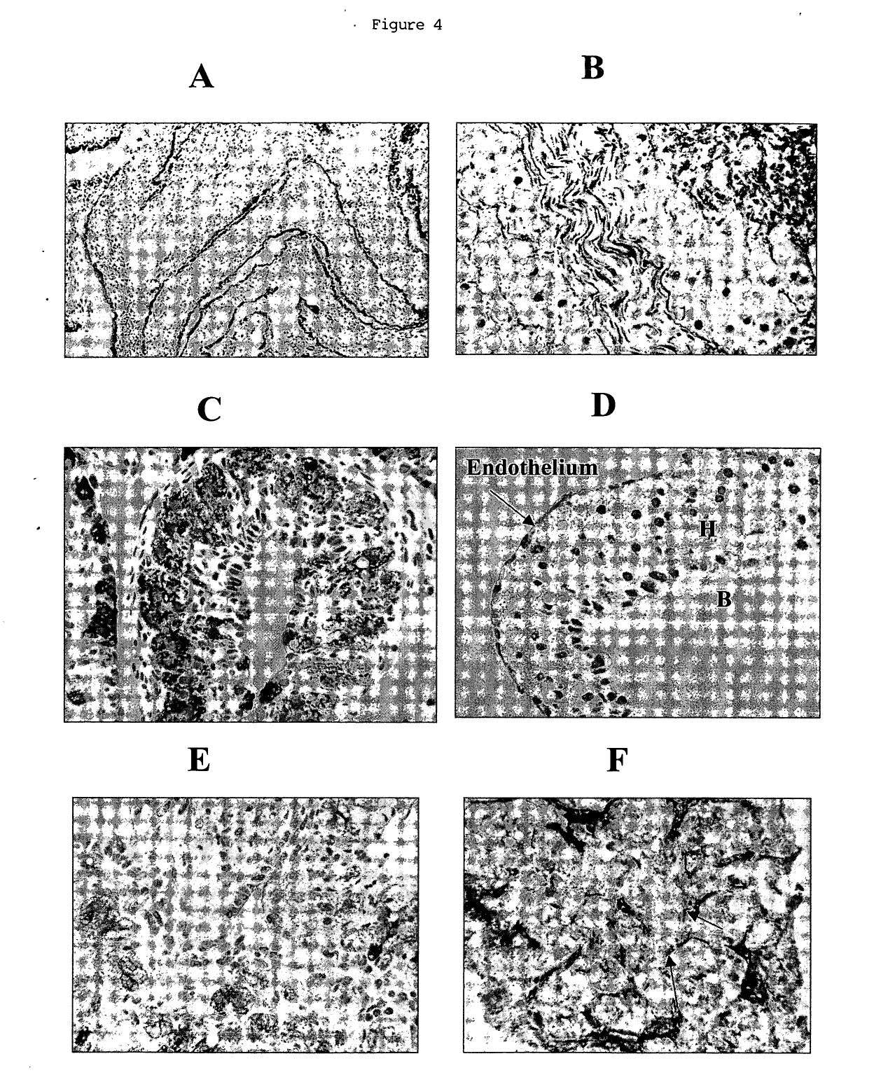

The superficial biliary epithelial cells were positive for cytokeratin 19, as expected and they appear as a linear brown staining on low power (FIG. 4A). Desmin, typically present in myofibroblasts and stellate cells, was seen in mesenchymal cells embedded in the connective tissue matrix and associated with presence of collagen bundles (FIG. 4B). HEPPAR antibody (Fiel M I, 1997, Mod Pathol 10:348-353) as well as antibody to cytochrome P-450 IIB1 stained hepatocytes positive, with occasional biliary epithelial cells also staining positive for the markers (FIG. 4, C and E, correspondingly). The endothelial cells in the basal surface were positive for factor VIII (FIG. 4D). Canaliculi stained positive for Mg++ ATPase (FIG. 4F, see arrows) (Hendrich S et al., 1987 Carcinogenesis 8:1245-1250).

Cellular Kinetics

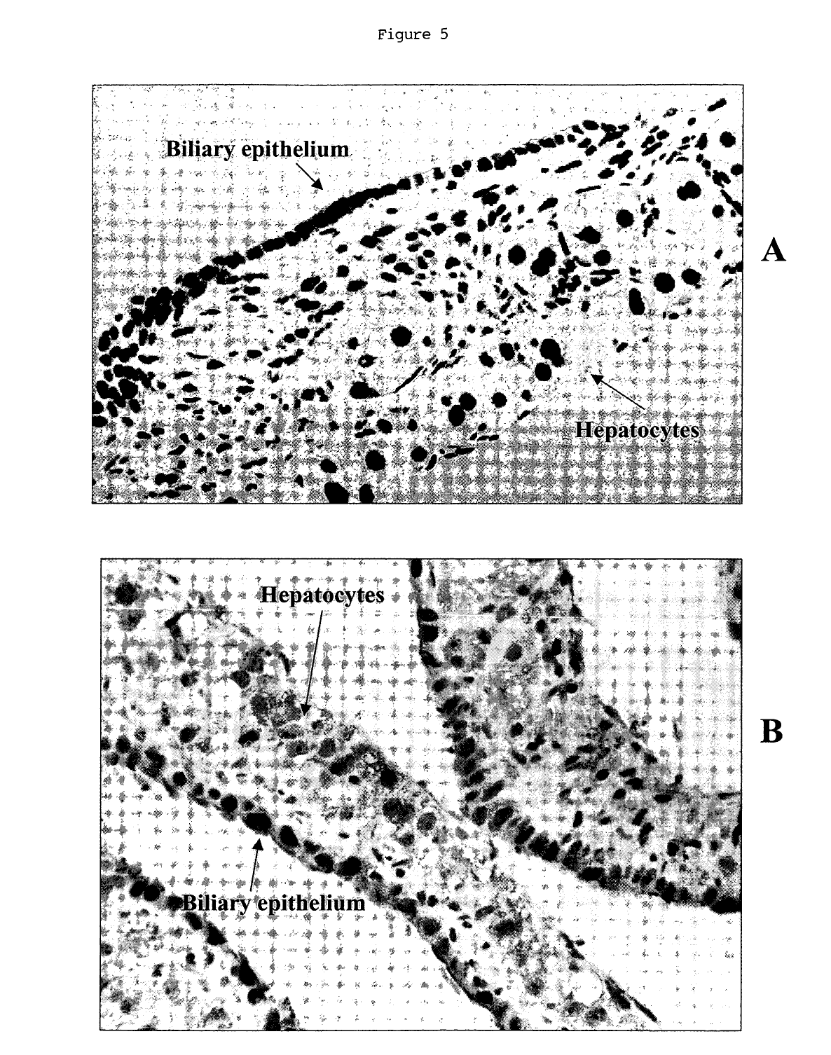

In the presence of HGF and EGF, most cells (>70% for each type) stained positive for PCNA (FIG. 5A). This indicates that most of the cells in the cultures are in the cell cycle. The antigen Ki-67 is typically expressed in cells actually in S phase. Less than 5% of the hepatocytes in the cultures stained positive for Ki-67 whereas >60% of the biliary epithelial cells were positive (FIG. 5B). A higher (>80%) PCNA labeling and a higher Ki-67 labeling were noted in all systems in which dexamethasone was not present (see below).

Influence of Growth Factors and Hormones on Tissue Organization

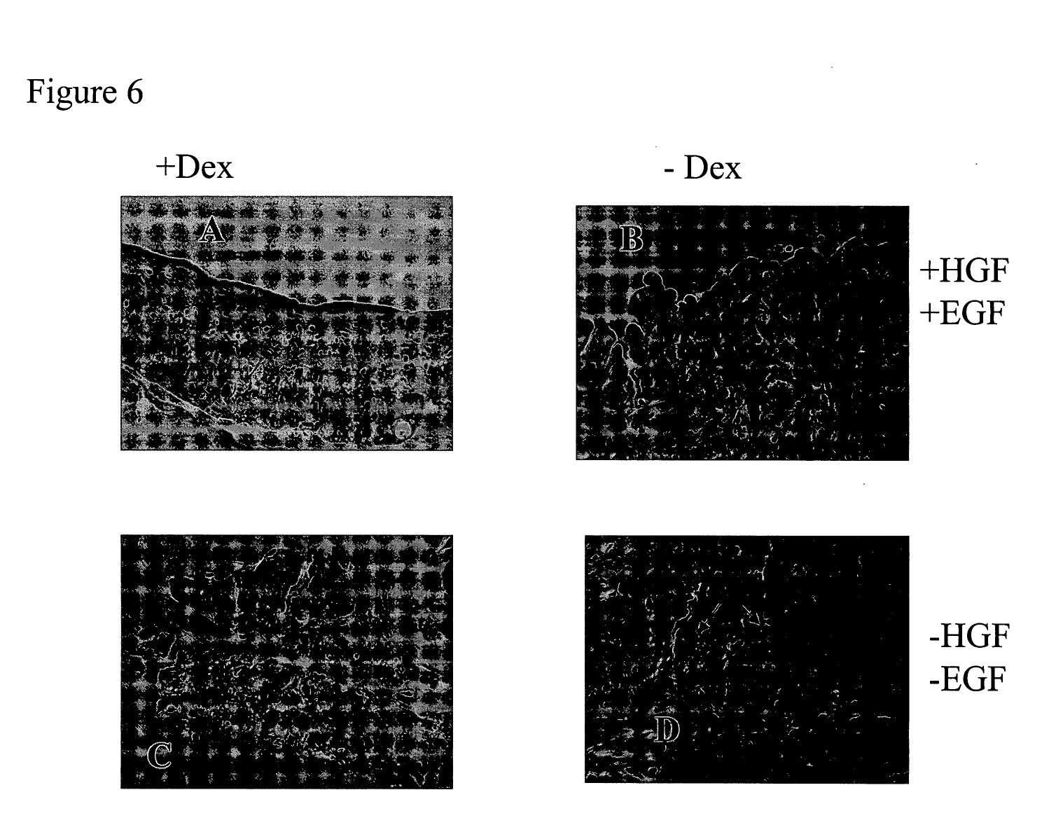

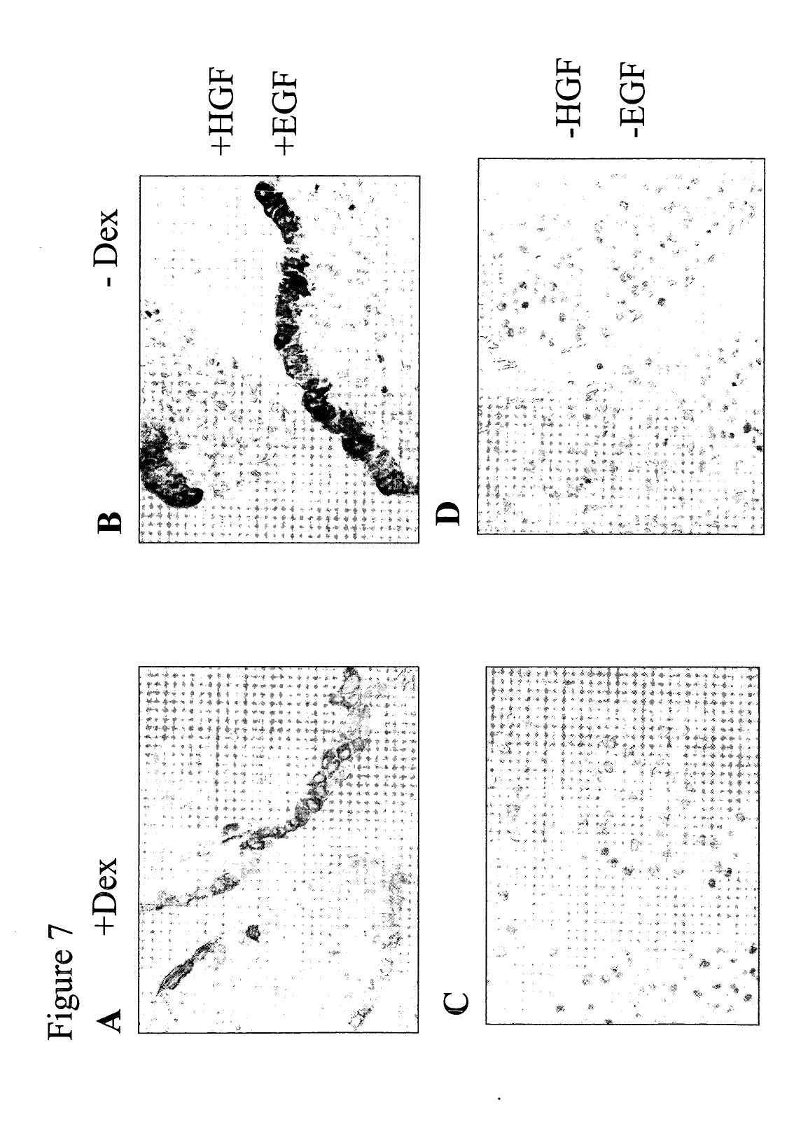

The results of these studies are shown in FIG. 6 (H&E stains) and FIG. 7 (cytokeratin 19 stain, as a marker for the biliary epithelium). The typical histology described above was seen in cultures maintained in the presence of dexamethasone, HGF, and EGF (FIGS. 6A and 7A) (please note that FIGS. 1B and 6A are identical, for comparison purposes). The histology of the cultures however was very much affected by selective elimination of these components.

Removal of EGF and HGF, Presence of Dexamethasone

Combined removal of these two growth factors resulted in elimination of the biliary epithelium in day 20 cultures. Hepatocytes were recognizable but small and remained negative for the HEPPAR and cytochrome P-450 IIB1 antigens. Many apoptotic hepatocytes were embedded in the histology of the cultures. No connective tissue development was noted.

Removal of Dexamethasone, Presence of HGF and EGF

There was an overall arrest in phenotypic maturation of hepatocytes. The cells resembled oval cells seen in rat liver in vivo. Some immature hepatocytes (<15% of the total) were positive for HEPPAR and cytochrome P-450 IIB1. Although cytokeratin 19 strongly labeled only the surface epithelium (FIG. 7B), there was no clear demarcation between the surface biliary epithelium and the underlying hepatocytes in H&E stains (FIG. 6B). There were no canalicular structures as demonstrable by Mg++ ATPase or electron microscopy. Connective tissue was present. Ki-67 labeling index was ˜10%.

Removal of Dexamethasone, HGF and EGF

The surface biliary epithelium was absent (FIG. 7D). Hepatocytes (FIG. 6D) appeared immature, similar to those seen in FIG. 6B. Some immature hepatocytes (<35% of the total) were positive for HEPPAR and cytochrome P-450 IIB1. Surprisingly, several mitoses and a high PCNA (>90%) and Ki-67 (25%) labeling index for hepatocytes were seen in these cultures. Connective tissue was present.

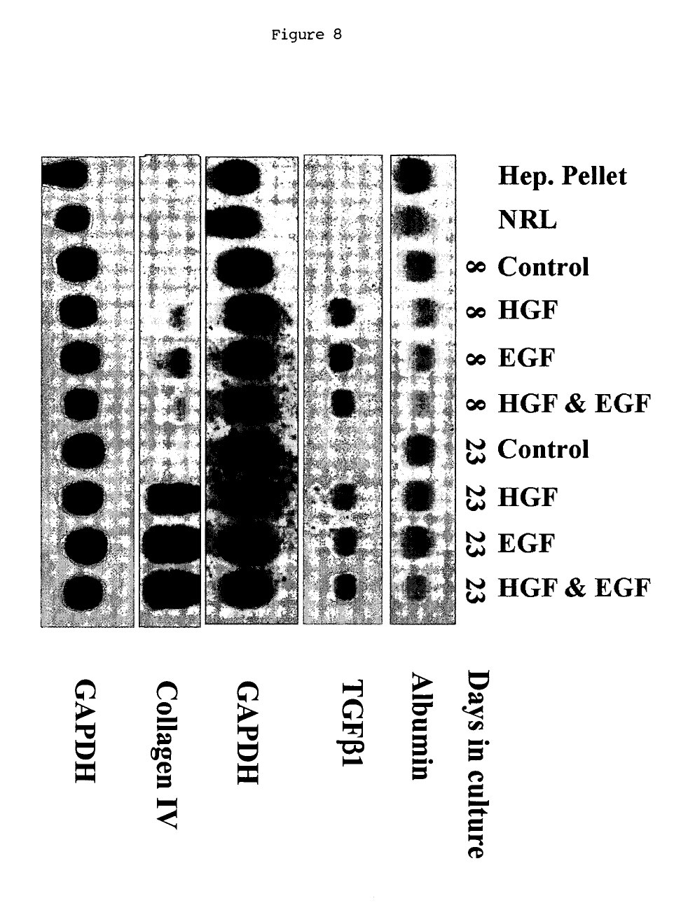

The combined results indicate that dexamethasone is required for the formation of fully mature, histologically recognizable, hepatocytes, distinct from the biliary layer. This is more apparent by simple histological analysis when HGF and EGF are present (compare FIG. 6A, B). When dexarnethasone alone is added, it inhibits cell proliferation and is associated with smaller atrophic hepatocytes. Thus, although dexamethasone is a modulator of hepatocyte differentiation, its effects vary depending on HGF, EGF, and perhaps other components of the medium. HGF and EGF are required for the appearance, maintenance, or growth of the biliary epithelium. Addition of either HGF or EGF alone restored formation of the biliary epithelium, but not to the full extent as seen when both growth factors were present. Connective tissue formation also depends on the presence of HGF and EGF. As mentioned above, when the nonparenchymal fraction isolated from collagenase perfusion of the rat liver was placed in culture in the absence of hepatocytes, and with the full complement of the HGF medium plus dexamethasone, HGF, or EGF, no growth of connective tissue elements or any tissue formation was noted. EGF or HGF alone restored some connective tissue formation in these cultures. EGF appeared more efficient in restoring connective tissue formation. The histological findings paralleled results from analysis of gene expression. FIG. 8 demonstrates expression of collagen type IV in cultures maintained in the presence of no growth factors (control), EGF alone, HGF alone, and EGF plus HGF. The strongest expression of collagen IV gene is seen in cultures maintained in the presence of EGF (alone or in combination with HGF). HGF alone also increased expression of type IV collagen above the control values at both day 8 and day 23 in culture, but to a lesser extent than EGF. Both growth factors however were equally efficient in inducing expression of TGF-β. In contrast, there were no apparent differences related to growth factors for albumin expression.

The practice of the present invention will employ, unless otherwise indicated, conventional techniques of cell biology, cell culture, molecular biology, recombinant DNA, and immunology, which are within the skill of the art. Such techniques are described in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Antibodies: A Laboratory Manual, and Animal Cell Culture (R. I. Freshney, ed. (1987); Culture of Animal Cells, A Manual of Basic Technique, 2d Ed., (R. I. Freshney, A. R. Liss, Inc., New York, 1987); Culture of Epithelial Cells (R. I. Freshney ed, Wiley-Liss, 1992), Embryogenesis in vitro: Study of Differentiation of Embryonic Stem Cells. Biol Neonate (Vol 67:77-83, 1995); Cell Therapy: Stem Cell Transplantation, Gene Therapy, and Cellular Immunotherapy (G. Morstyn & W. Sheridan eds, Cambridge University Press, 1996); and Hematopoietic Stem Cell Therapy, (E. D. Ball, J. Lister & P. Law, Churchill Livingstone, 2000).

Equivalents

While specific embodiments of the subject invention have been discussed, the above specification is illustrative and not restrictive. Many variations of the invention will become apparent to those skilled in the art upon review of this specification. The appendant claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entireties as if each individual publication or patent was specifically and individually indicated to be incorporated by reference. In case of conflict, the present application, including any definitions herein, will control.

The contents of each of the references cited in the present application, including publications, patents, and patent applications, are herein incorporated by reference in their entirety.

The present invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

We claim:

1. A method for generation cultures of small tissues comprising incubating cells from said small tissues in a horizontally-rotating vessel, wherein said vessel
(a) contains one or more growth agents that support the growth of said cells and
(b) is coated with extracellular matrix protein that promotes cell adhesion under conditions sufficient to allow for proliferation of said cells while retaining the function of said cells.

2. The method of claim 1 wherein said extracellular matrix protein is type I collagen.

3. The method of claim 1 further comprising culturing said cells in the presence of dexamethasone.

4. The method of claim 1 wherein said small tissue is adrenal tissue.

5. The method of claim 4 wherein said growth agents is transforming growth factor-

6. The method of claim 1 wherein said small tissue is bile duct epithelia.

7. The method of claim 6 wherein said growth agents are hepatocyte growth factor and epidermal growth factor.

8. The method of claim 1 wherein said small tissue is corneal tissue.

9. The method of claim 8 wherein said growth agent is epidermal growth factor.

10. The method of claim 1 wherein said small tissue is pituitary tissue.

11. The method of claim 10 said growth agent is growth hormone.

12. The method of claim 1 wherein said small tissue is thyroid tissue.

13. The method of claim 12 wherein said growth agent is from hypothalamic and pituitary extracts.

14. Small tissue culture derived from a method comprising incubating cells from said small tissues in a horizontally-rotating vessel, wherein said vessel
(a) contains one or more growth agents that support the growth of said cells and
(b) is coated with extracellular matrix protein that promotes cell adhesion and under conditions sufficient to allow for proliferation of said cells while retaining the function of said cells.

15. The small tissue culture of claim 14 wherein said extracellular matrix proteins is type I collagen.

16. The small tissue culture of claim 14 further comprising culturing said cells in the presence of dexamethasone.

17. The small tissue culture of claim 14 wherein said small tissue is adrenal tissue.

18. The small tissue culture of claim 17 wherein said growth agent is transforming growth factor β.

19. The small tissue culture of claim 14 wherein said small tissue is bile duct epithelia.

20. The small tissue culture of claim 19 wherein said growth agents are hepatocyte growth factor and epidermal growth factor.

21. The small tissue culture of claim 14 wherein said small tissue is corneal epithelia.

22. The small tissue culture of claim 21 wherein said growth agent is epidermal growth factor.

23. The small tissue culture of claim 14 wherein said small tissue is pituitary tissue.

24. The small tissue culture of claim 23 wherein said growth agent is growth hormone.

25. The small tissue culture of claim 14 wherein said small tissue is thyroid tissue.

26. The small tissue culture of claim 25 wherein said growth agents are from hypothalamic and pituitary extracts.

27. A method of providing small tissue function to a subject having a disorder of the small tissue comprising administering to said subject the small tissue culture of claim 14.

28. The method of claim 27 wherein the small tissue is adrenal tissue.

29. The method of claim 27 wherein the small tissue is bile duct epithelia.

30. The method of claim 27 wherein the small tissue is corneal.

31. The method of claim 27 wherein the small tissue is pituitary tissue.

32. The method of claim 27 wherein the small tissue is thyroid tissue.
